(19) United States
(12) Patent Application Publication
(10) Pub. No.: US 2025/0331887 A1
(43) Pub. Date: Oct. 30, 2025

(54) DEVICE AND METHOD FOR DEPLOYING DURA SHIELD

(71) Applicant: SurGen Tec, LLC, Boca Raton, FL (US)

(72) Inventor: Ashish Patel, Chicago, IL (US)

(21) Appl. No.: 18/644,529

(22) Filed: Apr. 24, 2024

(51) Int. Cl. A61B 17/34; A61B 17/00; A61B 17/02

(52) U.S. Cl. CPC A61B 17/3403; A61B 17/02; A61B 17/3421; A61B 2017/00367; A61B 2017/0042

(57) ABSTRACT

A method and apparatus introduced herein deploy a dura shield within a biological tissue during surgical procedures to protect the biological tissue. The method may comprise steps of puncturing a hole into the biological tissue at a desired location on the biological tissue by pushing a needle disposed within a cannula coupled to the housing, and pressing a button, coupled to an actuation plunger movably disposed within the housing, to cause forward movement of the actuation plunger, advance the dura shield guided by the cannula, and drive the dura shield through the hole.

### TECHNICAL FIELD

The present disclosure is generally related to medical devices and more specifically retractors and/or barriers used during surgery.

### BACKGROUND

Dura is a membrane that surrounds the brain and spinal cord, protecting the central nervous system. The dura has several layers containing cerebrospinal fluid and nerves.

### SUMMARY

Dura shields in accordance with examples are disclosed. In one example, a dura shield includes a shield and/or barrier configured to protect a patient's dura and or/nerves during an interbody procedure, a tether coupled to the shield and configured to position the shield, and an anchor coupled to the shield and configured to attach to a surface within the patient during the interbody procedure.

In yet another example, the shield is constructed using a material selected from the group consisting of plastic, rubber, sponge, neoprene, polyethylene fibers, and Kevlar.

In still another example, the shield comprises a first layer constructed using a first material selected from the group and a second layer constructed using a second material selected from the group, the first material differing from the second material.

In yet still another example, the first layer provides an abrasion-resistant surface.

In yet another additional example, the second layer provides a padded surface.

In still another additional example, the tether is located between the first layer and the second layer.

In yet still another additional example, the shield comprises an antibacterial treatment.

In yet another example, the shield comprises a soothing treatment.

In still another example, the surface is selected from the group consisting of an annulus of a spinal disc, a spinal disc, and a bone.

In yet still another example, the anchor is selected from the group consisting of a staple, a hook, a screw, a tag, a suture, and an adhesive.

In yet another additional example, the tether is constructed from a material selected from the group consisting of a suture, a plastic, a rubber, and a metal.

In still another additional example, the dura shield comprises an unopened state where the shield is rolled around the tether and an opened state where the shield is unrolled and the tether can be used to adjust the positioning of the shield.

In yet still another additional example, the anchor is attached to the surface when the dura shield is in the opened state.

In yet another example, the dura shield includes an applicator configured to hold the dura shield when the dura shield is in the unopened state or the open state.

In still another example, the applicator is configured to transition the dura shield from the unopened state to the opened state by unrolling the shield from around the tether as the applicator is withdrawn from the surface.

Yet another example includes a method for attaching a dura shield including inserting, into a piston of an applicator, a dura shield in an unopened state, wherein an anchor of the dura shield extends beyond a first end of the piston, placing the anchor on a surface, actuating a trigger of the applicator to cause the piston to move toward an applicator tip of the applicator and to attach to the surface, and deploying the dura shield by withdrawing the piston from the applicator tip, wherein withdrawing the piston causes the dura shield to move from the unopened state to an opened state.

In yet another example, the method further includes manipulating a location of the dura shield by moving a tether of the dura shield using the applicator.

In still another example, causing the anchor to attach to the surface comprises compressing a staple.

In yet still another additional example, causing the anchor to attach to the surface comprises turning a screw.

In yet another additional example, causing the plastic tag to attach to the surface comprises inserting and deployment of a tag or anchor

Still another example includes a dura shield including a tether, a shield configured to protect a patient's dura during an interbody procedure and coupled to the tether, the shield comprising a rolled state where the shield is rolled around a tether and an unrolled state wherein the shield is unrolled and the tether can be used to adjust positioning of the shield, a staple or tag coupled to the shield and configured to attach to an annulus of a spinal disc within the patient during the interbody procedure, and an applicator configured to hold the dura shield when the shield is in the rolled state and configured to manipulate the shield from the rolled state to the unrolled state. Other objects, advantages and novel features, and further scope of applicability of the present disclosure can be set forth in part in the detailed description to follow, and in part can become apparent to those skilled in the art upon examination of the following, or can be learned by practice. The objects and advantages of the disclosure can be realized and attained by means of the instrumentalities and combinations particularly pointed out in the claims.

### BRIEF DESCRIPTION OF THE DRAWINGS

The description can be more fully understood with reference to the following figures, which are presented as examples and should not be construed as a complete recitation of the scope, wherein:

FIG. 1 is a line drawing of an unopened dura shield in accordance with an example;

FIG. 2A is a line drawing of an opened dura shield in accordance with an example;

FIG. 2B is a line drawing of an opened dura shield in accordance with an example;

FIG. 3A is a conceptual line drawing of a dura shield coupled to an annulus of a spinal disc in accordance with an example is shown;

FIG. 3B is a line drawing of a dura shield in vivo in accordance with an example;

FIG. 4 is a line drawing of an applicator for a dura shield in accordance with an example; and

FIG. 5 is a line drawing of an applicator for a dura shield in accordance with an example.

FIG. 6 shows an example of a dura shield.

FIG. 7 shows an example of a dura shield applicator.

FIG. 8A shows an example of a dura shield applicator prior to loading a dura shield.

FIG. 8B shows an example of a dura shield applicator loaded with a dura shield.

FIG. 8C and FIG. 8D show examples of operations of a dura shield applicator.

FIG. 9 shows an example of a dura shield applied in Lateral Lumbar Interbody Fusion (LLIF) or Oblique Lateral Lumbar Interbody Fusion (OLLIF) procedures.

FIG. 10 shows an example of a dura shield applied in Anterior Lumbar Interbody Fusion (ALIF) procedure.

### DETAILED DESCRIPTION

Turning now to the drawings, dura shields in accordance with examples are disclosed. Interbody cages are typically titanium structures surgically inserted in the spinal disc space. The interbody cage is usually porous and permit bone grafts to grow from the vertebral body through the cage and into the next vertebral body. During surgery, such as spinal surgeries, several elements are under risk for injury including, but not limited to, nerves and blood vessels. While the following is described with respect to spinal surgeries, dura shields can also be used for several applications occurring during a wide variety of medical procedures.

Interbody fusion surgeries, such as lumbar fusions, creates connections between adjoining vertebra, thereby eliminating any movement between the bones. A variety of lumbar fusion surgeries are commonly utilized, such as transforaminal lumbar interbody fusion (TLIF), posterior lumbar interbody fusion (PLIF), anterior lumbar interbody fusion (ALIF), lateral interbody fusion (LLIF), and the like. TLIF fuses the anterior (front) and posterior (back) columns of the spine, where the anterior portion of the spine is stabilized by the bone graft and interbody spacer and the posterior column is locked in place with pedicle screws, rods, and/or bone graft. PLIF includes approaching the spine through the back and inserting a cage made of either allograft bone or synthetic material (e.g. titanium) directly into the disc space of the spine. ALIF and LLIF are similar to PLIF in that these procedures include inserting a cage made of either allograft bone or synthetic material (e.g. titanium) directly into the disc space of the spine; however, in ALIF the spine is approached through the peritoneum while in LLIF a retroperitoneal approach to the spine is utilized.

For TLIF, the surgeon may approach the TLIF through an open procedure, minimally invasive tubular procedure, or minimally invasive pedicle screw-based retractor. There is bony work that is done initially either a laminotomy or a facetectomy to remove the bone that is in the way to enter the spinal canal. Once the bone is removed and there is a pathway into the spinal canal, typically the dura is seen, and/or traversing and exiting nerve root. The dura shield may be used to protect the dura, and/or the existing nerve root. In the first step, the dura will be retracted to gain access to some of the annulus or the PLL ligament. At this point, the dura shield is applied to attach to the annulus or the adjacent bone if the annulus is very thin and the disc space is degenerated. The method for application of the dura shield is described in the patent already when an attachment is with an anchor, a tag, a suture, a screw, or a staple. Once the attachment is conducted, the shield is deployed, and that may be draped over the dura or the exiting nerve root to retract it out of the way and create a pathway to the disc space where the nerves are protected at which point the rest of the discectomy and the inter-body procedure may be conducted.

The second method for TLIF is after the facetectomy is done and the initial discectomy is done, where the annulus is incised and disc material is removed. The dura shield may be applied after a preliminary discectomy is conducted to visualize the disc space and the annulus better. The dura shield may be deployed similarly as before just after the initial discectomy is completed. At the end of the discectomy and the inter-body procedure, the dura shield may be removed with the removal tool or with Kerrison or a knife to cut the anchoring device and remove the dura shield so it does not remain in the body. That concludes the TLIF procedure.

For the ALIF procedure, after the anterior approach is conducted to anterior lumbar disc spaces, the vessels are mobilized. Either the access surgeon or the spine surgeon would anchor, through the method that was described before, the dura shield to the lateral disc space. The purpose of the dura shield in this context would be to help gentle retraction of the mobilized vessels, whether anchored to the disc or anchored to bone. This may create a pathway to the disc space, or the adjacent bone with the vessels retracted to reduce or minimize the risk of vascular injury. Similarly, the dura shield may be anchored either before any discectomy or just after a preliminary discectomy. Blood vessel retraction may be done by only the dura shield itself or with the use of the dura shield and a formal ALIF retractor in front of the dura shield Again the dura shield may be removed after the inter-body procedure is completed so it is not to have any material remain in the body. This concludes the ALIF procedure.

For the LLIF, the dura shield may be used to set up the anterior and posterior margins of the working corridor. The dura shield may be anchored to the disc space or the adjacent bone. The dura shield may be used to retract muscles, femoral nerve, and nerve roots posteriorly or anteriorly, setting up a working corridor. Again the dura shield may be removed at the end of the procedure so there is no material remains. This concludes applications of dura shield in TLIF, ALIF, and LLIF.

Further, there are other methods where the dura shield may be used, including in anterior cervical discectomy and fusion, where the dura shield is utilized to retract the esophagus or trachea after anchoring it to the bone and disc or to retract the carotid artery and the venous network. In other iterations, the dura shield may be used to retract lung tissue when navigating through the thoracic cavity, after anchoring the dura shield to bone or disc in the spine. This is particularly relevant during corpectomy or vertebral column resection (VCR) for fracture work or tumor removal. Additionally, in iterations like pedicle subtraction osteotomy or VCR performed from a posterior approach, the dura shield may be positioned to anchor to disc or bone tissue within the spine to retract the dura mater or blood vessels. In the context of orthopedics or general surgery, the dura shield is attached to distal bone tissue and is used to retract nerve tissue, blood vessels, or other delicate tissues that are susceptible to injury during surgical procedures. The composition of the dura shield and its tether may comprise materials like cortex, rubber, silicone, plastics, and metals. The shield itself may be designed from various materials, serving as a protective device for fragile tissues during surgery. The tether may be made from suture material, plastic, or silicone to securely anchor the dura shield. The anchoring device may be a screw, suture, curved or straight tag made of plastic or metal, a staple device, or a suturing device, and it may be anchored to the disc, spinal tissue, or adjacent bone at the spinal level, allowing for the independent retraction of sensitive tissues like nerves or blood vessels. The dura shield may be applicable in spine surgery, neurosurgery, and general orthopedic surgery. It may come as a reusable kit, wherein the deployment device may be used multiple times, and the shield, being a single-use unit, is disposable. Alternatively, the entire kit, including the deployment device, dura shield, and tethering device, may be designed for one-time use as a disposable kit.

When an interbody fusion is performed, the dura (which includes the lumbar nerve roots) is mobilized and retracted medially. The exiting nerve root at times is retracted superiorly or laterally as needed. This then uncovers the spinal disc space that will be accessed for interbody fusion. The dura is typically retracted with a metallic nerve root retractor held for the duration of the interbody fusion, usually by an assistant. Not only does this typically leave the assistant unable to perform other duties, the retraction of the dura can lead to a variety of problems, such as the dura being impacted and/or damaged during the surgical procedure. This can lead to nerve damage and/or the leaking of spinal fluid occurring during surgery, requiring additional procedures to correct, potential complications from the surgery, and additional recovery time for the patient.

Dura shields disclosed herein are designed to replace the metallic dura retractor typically used during interbody fusion surgeries. A dura shield provides a barrier between instruments working in the spinal disc space and the dura which is at risk for injury and to define the working corridor during these procedures. The dura shield can be anchored so that it protects the dura from impacts and/or abrasion. The dura shield can be anchored to the annulus of the spinal disc through a staple, a plastic or metal tag or other attachment device. In the event of minimal annulus, the dura shield can also be anchored to the bone in or outside the disc space. The dura shield can include a tether that can be used to position the dura shield and/or hold the dura in place. In this way, the dura shield disclosed herein can replace both the dura retractor and the assistant during the operation while adding additional safeguards to the dura, reducing complications, and reducing recovery time. This ‘dura retraction’ can be automated with the dura shield versus the standard practices typically employed.

In a variety of examples, the dura shield includes one or more sensors and/or sensing surfaces. These sensors and/or sensing surfaces can be detected by a variety of devices, such as surgical robots and spinal navigation devices, while the device is performing one or more actions during the operation. The device can use the sensors and/or sensing surfaces to identify the range in which the device can move and/or perform actions during the operation.

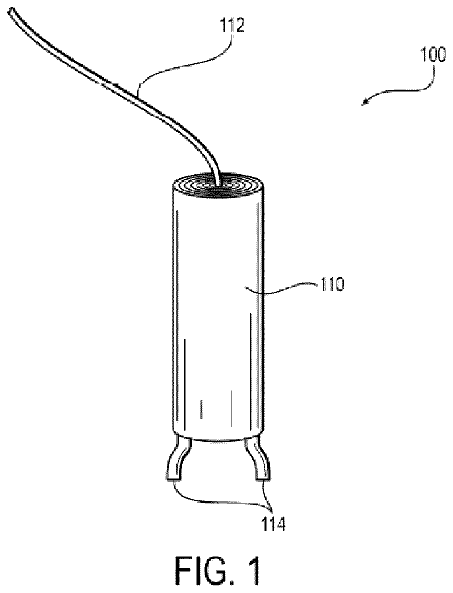

Turning now to FIG. 1, a line drawing of an unopened dura shield in accordance with an example is shown. Internal portions of the device are shown in dotted lines. The dura shield 100 includes a shield 110, a tether 112, and an anchor 114. In the unopened state, the shield 110 is wrapped around the tether 112 and/or anchor 114. The shield 110 can be made of any material, such as plastic, rubber, sponge, neoprene, polyethylene fibers, Kevlar, and/or any other material that provides padding and/or abrasion resistance as appropriate. In several examples, the shield 110 is made from a single material. In a variety of examples, the shield 110 includes two or more layers. In many examples, one or more of the layers of the shield 110 are made from different materials. For example, the shield 110 can include a top layer made from an abrasion-resistant material and a bottom layer that is made from a padded material. In a number of examples, the shield 110 utilizes multiple materials in a single layer. For example, the shield can have a lower portion made from rubber and an upper portion made from a padded material, such as a sponge and/or neoprene.

The tether 112 can be used to position the shield 110 once deployed and/or hold back a portion of the body, such as the dura. In several examples, the tether 112 is attached to the shield 110 and/or anchor 114. The tether 112 can be constructed out of any material, such as plastic, rubber, and/or metal, as appropriate. The tether 112 may be affixed to an external surface and/or weighted to cause the shield 110 to stay in contact with a desired surface once deployed.

The anchor 114 can be used to couple the shield 110 and/or tether 112 to one or more bodies or surfaces within a surgical space. For example, during a spinal surgery, the anchor 114 can be attached to an annulus of a spinal disc. However, the anchor 114 can attach to any surface, such as directly to the bone of the spinal disc, as appropriate. The anchor 114 can include, but is not limited to, a staple, a hook, a screw, a tag, an adhesive, and the like. The anchor 114 can be constructed out of any material that is suitable for interaction with the corresponding attachment surface. For example, the anchor 114 can be constructed using a ductile material that allows for the anchor 114 to wrap around an annulus of a spinal disc. In another example, the anchor 114 can be a staple constructed using a metal that is formulated to allow penetration into bone without being so hard that it causes the bone to break. In several examples, the anchor 114 is constructed using a plastic that maybe a tag that is used to puncture the disc and/or bone and anchor the dura shield in place. In many examples, a first end of the anchor 144 is coated in an adhesive that couples the anchor 114 to a bone and/or any other surface in a body. The adhesive can be any non-permanent adhesive as appropriate. In a variety of examples, the adhesive is temperature-dependent such that the adhesive adheres to a surface, such as a disc or bone, at typical body temperatures and releases at a temperature above normal body temperature. Any temperature differential, such as 10 degrees, between the temperature at which the adhesive will couple the anchor 114 to the surface and the temperature at which the adhesive will release the anchor 114 from the surface can be used as appropriate.

A variety of treatments and/or coatings can be applied to the shield 110, tether 112, and/or anchor 114 as appropriate. For example, antibacterial and/or soothing compounds can be applied to the shield 110. These compounds can be used to treat the shield 110 to reduce the risk of infections and complications from contaminants during surgery.

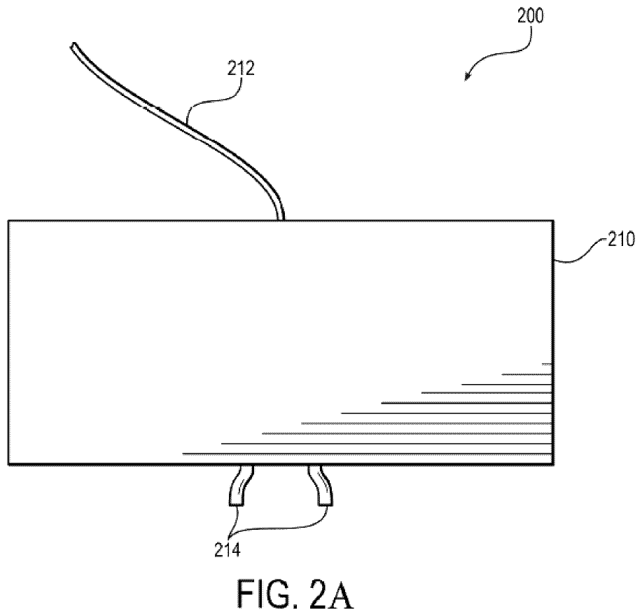

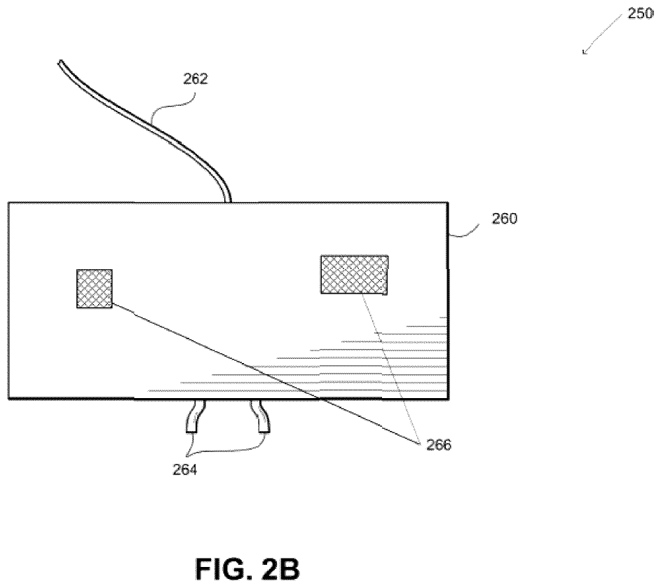

Turning now to FIG. 2A, a line drawing of an opened dura shield in accordance with an example is shown. Internal portions of the device are shown in dotted lines. The dura shield 200 includes a shield 210, a tether 212, and an anchor 214. In the opened state, the shield 210 is unrolled and lies substantially in contact with a surface. For example, the shield 210 can be substantially in contact with the retracted dura after the dura has been opened during an interbody surgery. The shield 210 may be oriented in any orientation relative to the spine depending on the specific incisions made to open and retract the dura from the spinal disc(s) being targeted during the surgery. This can be placed for a cervical, thoracic, and/or lumbar procedures to retract and protect structures that maybe injured during the interbody work. This includes the dura and nerve root(s) during TLIF procedure, the big blood vessels during ALIF, and/or lumbar plexus nerves and psoas muscle during the LLIF procedure, or the lung during LLIF procedures in the thoracic spine.

The dura shield can be packaged such that is automatically deploys from an unopened state to an opened state once the anchor 214 is affixed to a body and the dura shield 200 is removed from an applicator. In a variety of examples, the shield 210 is manually unrolled onto the surface. Once unrolled, the tether 212 can be used to maintain the shield 210 in contact with the surface. In several examples, the tether 212 maintains the shield 210 in movable contact with the surface such that the tether 212 can be used to adjust the positioning of the shield 210 with respect to the surface.

Turning now to FIG. 2B, a line drawing of an opened dura shield with sensor devices in accordance with an example is shown. The dura shield 250 includes a shield 260, a tether 262, an anchor 264, and one or more sensor devices 266. In the opened state, the shield 260 is unrolled and lies substantially in contact with a surface. For example, the shield 260 can be substantially in contact with the retracted dura after the dura has been opened during a spinal surgery. The shield 260 may be oriented in any orientation relative to the spine depending on the specific incisions made to open and retract the dura from the spinal disc(s) being targeted during the surgery. The dura shield can be packaged such that is automatically deploys from an unopened state to an opened state once the anchor 264 is affixed to an attachment surface and the dura shield 250 is removed from an applicator. In a variety of examples, the shield 260 is manually unrolled onto the surface. Once unrolled, the tether 262 can be used to maintain the shield 260 in contact with the surface. In several examples, the tether 262 maintains the shield 260 in movable contact with the surface such that the tether 262 can be used to adjust the positioning of the shield 260 with respect to the surface.

The one or more sensor devices 264 can be located in a variety of locations within dura shield 250. As shown, the dura shield 250 includes two sensor devices located on a first end and a second end of shield 260. However, any number of sensors devices can be located in dura shield 250, and the sensor devices can be located within shield 260, tether 262, and/or anchor 264 as appropriate. In many examples, the sensor devices 264 cover the entire surface (or, when the shield 260 includes multiple layers, an entire layer) of the shield 260.

The sensor devices 264 can provide an indication regarding the location of the dura shield 250 to a variety of surgical devices, such as robotic surgical devices and spinal navigation devices. As the dura shield 250 is located adjacent to sensitive structures within an operating environment, such as nerves and blood vessels, the sensor devices 264 can provide the devices with an indication of the contours of the effective borders of the surgical area within the operating environment. In this way, the sensor devices 264 can provide feedback to the surgical devices such that the devices can limit their range of motion to an area within the region protected by the dura shield 250.

The sensor devices 264 can be made from a variety of materials. In many examples, the sensor devices 264 include a colored and/or reflective surface on the top of shield 260 that can be detected using a variety of sensors, such as light sensors and/or color sensors. For example, the surface of shield 260 can be colored bright green (or any other color that does not naturally occur within the operating environment) that can be detected by a sensor in a surgical device. In a number of examples, the color and/or reflectivity of the shield 260 is such that it can be detected even if the surface of shield 260 is covered in material deposited on shield 260 during a surgical procedure. In several examples, sensor devices 264 include low-power radio devices, such as a Bluetooth low energy devices, radio frequency identification (RFID) tags, and/or near field communication (NFC) tags. These low-power radio devices can include a microprocessor and/or an antenna that is powered by an interrogator located in the surgical device. When activated, the sensor devices 264 can indicate that the surgical device is located within a threshold distance of the dura shield 250. In a number of examples, the sensor devices 264 include temperature and/or blood flow sensors (or any other sensors) that can monitor conditions of the patient during the surgical procedure. In a variety of examples, the dura shield 250 includes an energy harvester, such as a harvester that converts heat to electricity, to power the sensor devices 264. The sensor devices 264 can transmit the sensor data to the surgical devices when the surgical device is within transmission range of the sensor devices 264. The dura shield 250 can also include energy storage devices, such as capacitors, to store energy generated by the energy harvester and/or to power the sensor devices 264.

Although a variety of dura shields are shown and described with respect to FIGS. 2A-B, any of a variety of constructions, including those that utilize sensors located externally to the shield, can be utilized in accordance with examples.

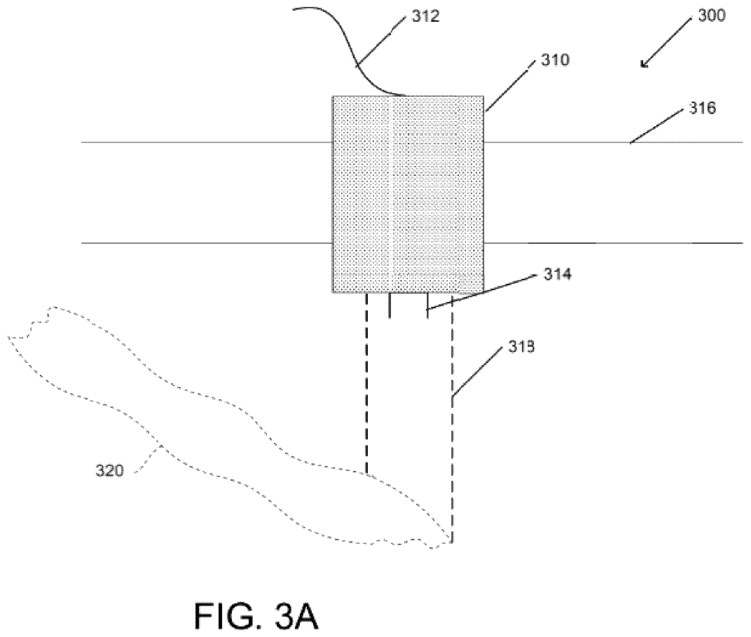

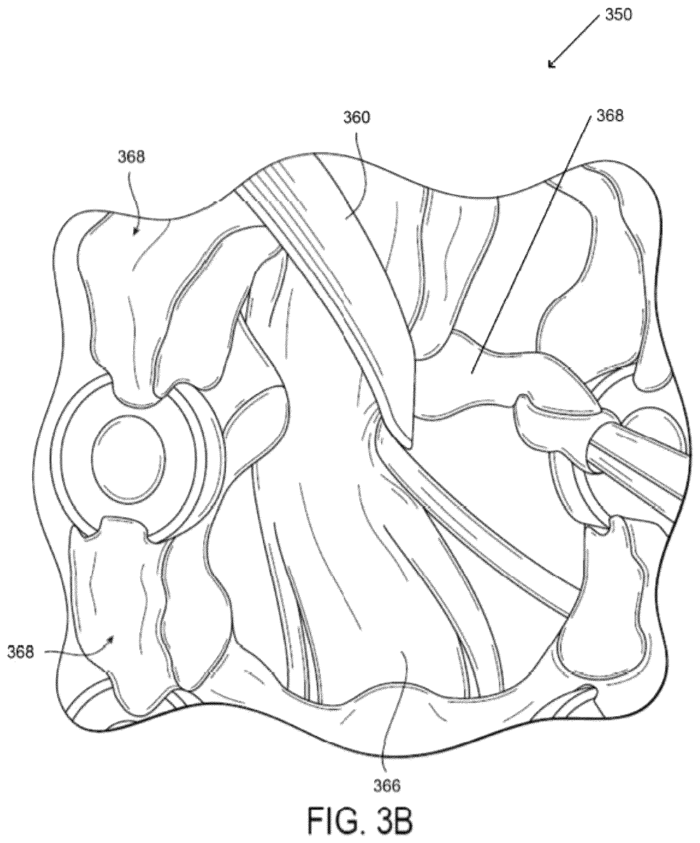

Turning now to FIG. 3A, a conceptual line drawing of a dura shield coupled to an annulus of a spinal disc in accordance with an example is shown. Portions of a body in which the dura shield may be attached are shown in dotted lines. The operating environment 300 includes a dura shield having a shield 310, tether 312, and anchor 314. The shield is deployed on dura 316 and the anchor 314 is coupled to an attachment surface 318. The attachment surface 318 is preferably an annulus of a spinal disc, but can be a spinal disc and/or any other surface within the body as appropriate to specific applications. Also shown is exiting nerve root 320. The shield 310 is opened and lies substantially in contact with to dura 316. In this way, the shield 310 protects the dura 316 from impact and/or abrasion. The tether 312 can be located within the shield 310, on top of the shield 310, and/or under the shield 310. In several examples, the tether 312 is located between the shield 310 and the dura 316. The tether 312 can be used to adjust the positioning of the shield 310 with respect to the dura 316.

Turning now to FIG. 3B, a line drawing of a dura shield in vivo in accordance with an example is shown. The operating environment 350 includes a dura shield 360 deployed on dura 366 and coupled to an attachment surface. A variety of attachment surfaces 368 are shown, including an annulus of a spinal disc and a spinal disc. However, any other surface within the body can be used as an attachment surface 368 as appropriate to specific applications. The dura shield 360 is opened and lies substantially in contact with to dura 366. In operating environment 350, the dura shield 360 is being used to adjust the location of dura 366 within the operating environment 350.

Although a variety of locations and attachments of dura shields are shown and described with respect to FIGS. 3A-B, it should be noted that any other positioning and/or attachment of a dura shield within an operating environment can be used in accordance with examples.

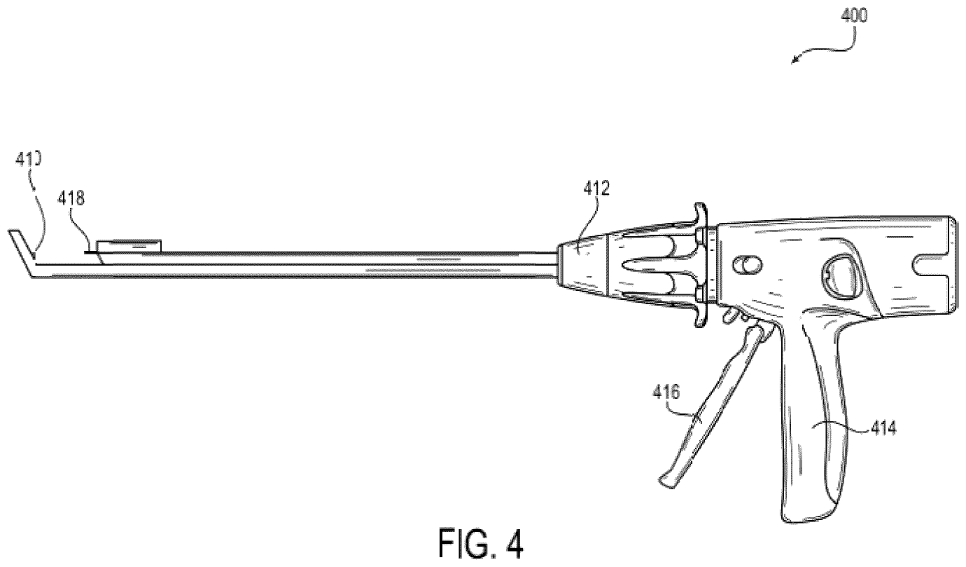

Turning now to FIG. 4, a line drawing of an applicator for a dura shield in accordance with an example is shown. Internal portions of the device are shown in dotted lines. The applicator 400 includes an applicator tip 410, a body 412, a handle 414, a trigger 416, and a piston 418. As shown in FIG. 4, the applicator 400 is in an unloaded state. The applicator tip 410 and/or body 412 can be dimensioned to accept a dura shield in an unopened state. The handle 414 can be used to hold the applicator 400, and the trigger 416 can be used to push piston 418 (or other structure) holding a dura shield towards the tip 410. The applicator tip 410 can cause an anchor of a dura shield to be affixed to a surface as described herein. In several examples, the applicator tip 410 is angled and can be used to place a dura shield relative to an attachment surface. When the piston 418 is brought into contact (or close to) the applicator tip 410, an anchor of the dura shield can be affixed to the attachment surface. In many examples, piston 418 can be actuated using the trigger 416 via one or more linkages in the body 412. Once affixed, the applicator 400 can be withdrawn, leaving the affixed dura shield to the surface. The applicator tip 410, piston 418, and/or body 412 can be shaped to cause the dura shield to transition from a closed state to an open state as the applicator 400 is withdrawn. In a variety of examples, the geometry of the applicator tip 410 is dimensioned to manipulate the position of the dura shield as the applicator 400 is withdrawn.

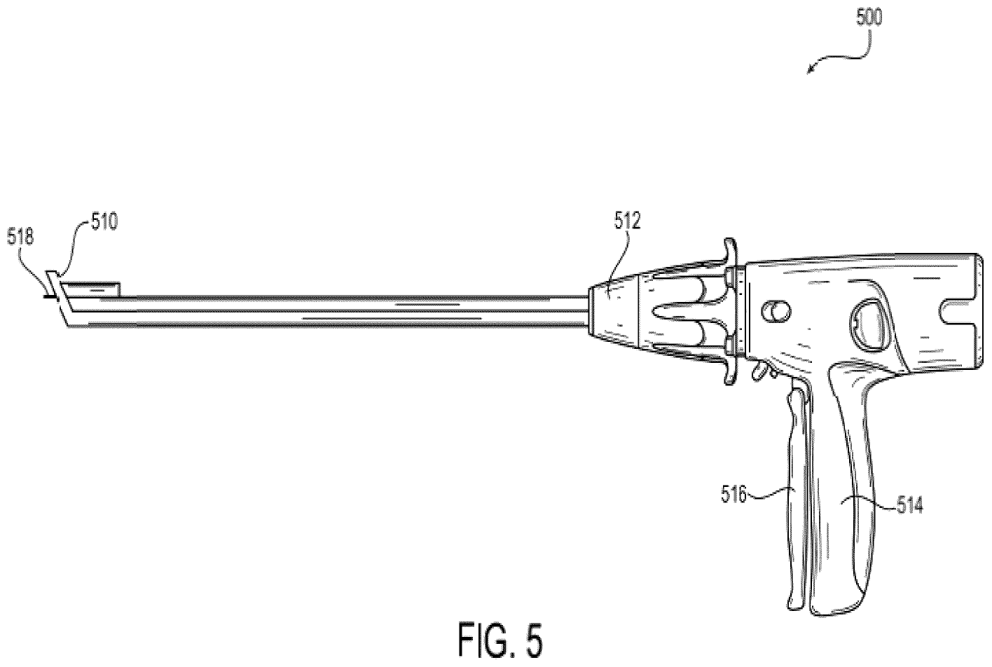

Turning now to FIG. 5, a line drawing of an applicator for a dura shield in accordance with an example is shown. Internal portions of the device are shown in dotted lines. The applicator 500 includes a dura shield 518, an applicator tip 510, a body 512, a handle 514, and a trigger 516. As shown in FIG. 5, the applicator 500 is in a loaded state. The dura shield 518 can be partially and/or fully contained within the applicator tip 510 and/or body 512. In several examples, an anchor of the dura shield 518 is contained external to the applicator 500, while the shield and tether of the dura shield are contained within the applicator tip 510 and/or body 512. The anchor of the dura shield 518 can be placed in contact with a surface. The handle 516 can be released, causing a piston to withdrawn from the applicator tip 510 and thereby deploying the dura shield 518 as described herein. In many examples, the dura shield is deployed as the applicator 500 is removed from the attachment surface.

Although a variety of applicators are shown and described with respect to FIGS. 4 and 5, a variety of other applicators, including those where the trigger is located away from the handle when the applicator is in a loaded state and the trigger can be squeezed in order to deploy the dura shield, can be used in accordance with examples.

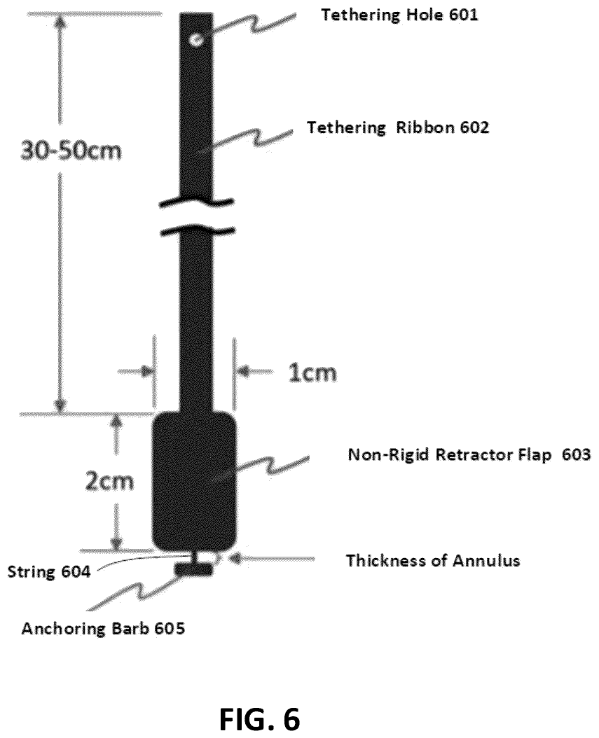

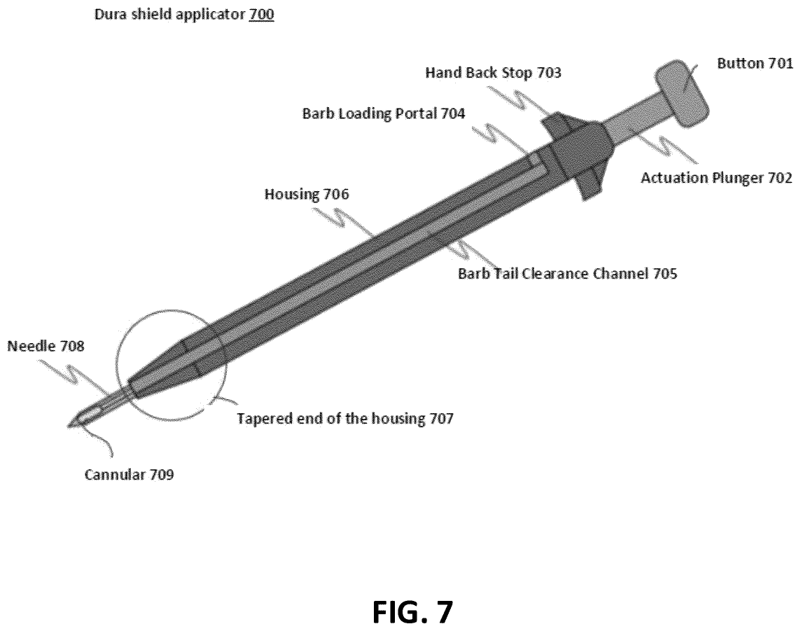

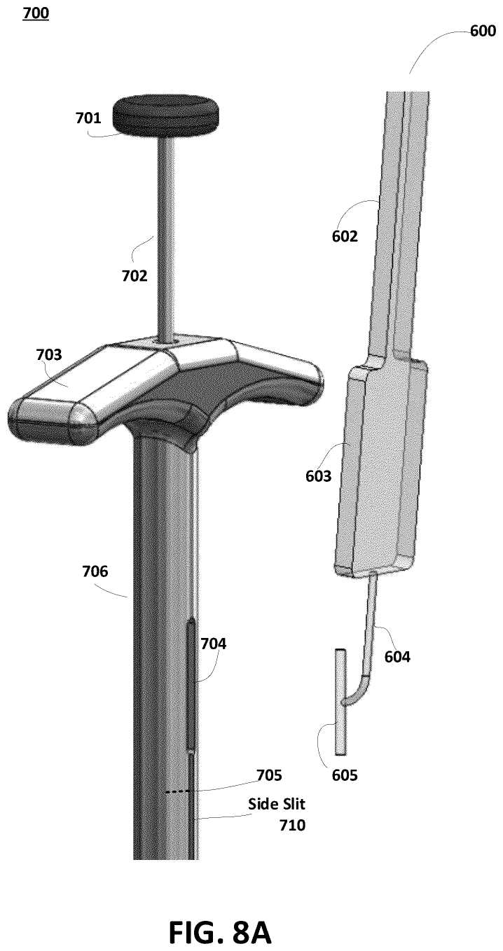

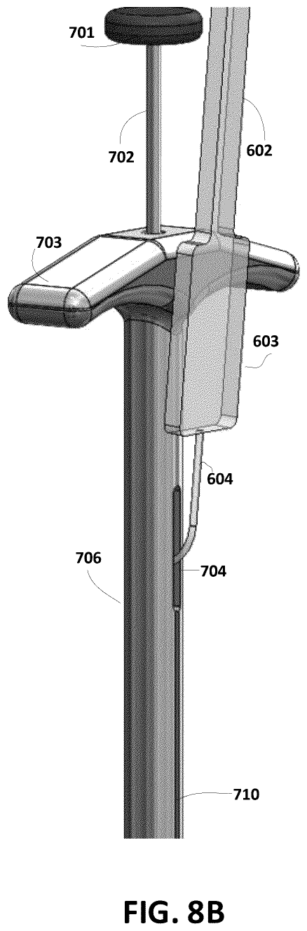

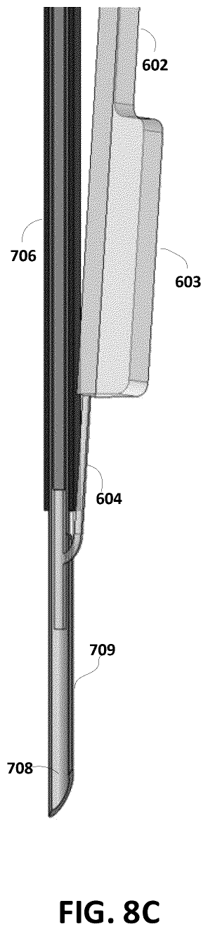

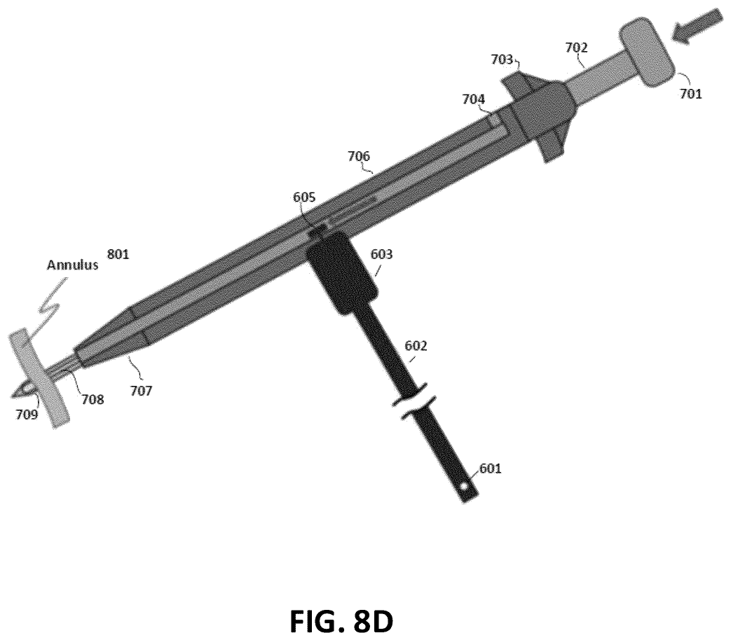

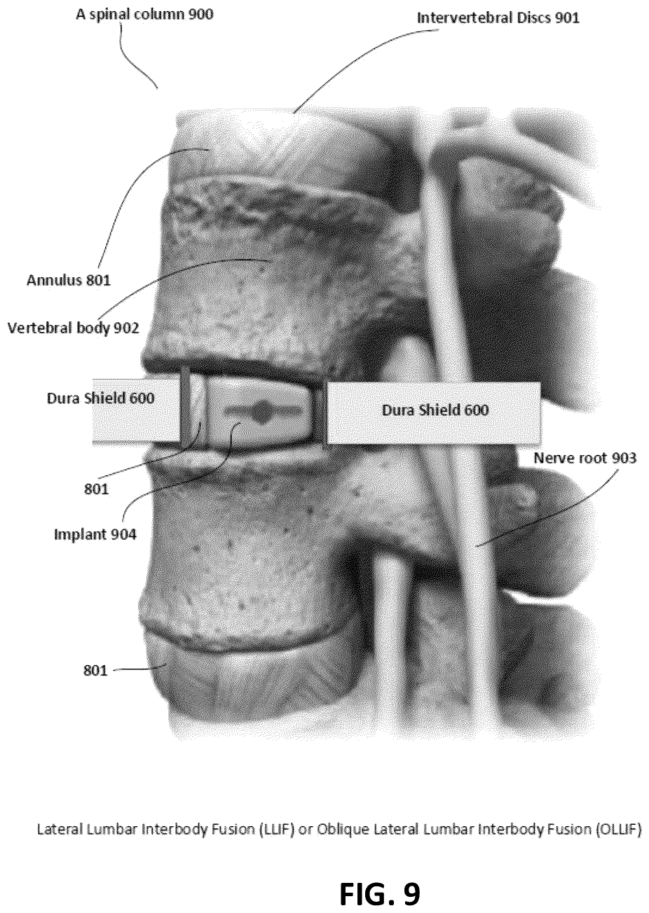

FIG. 6 shows an example of a dura shield 600. The dura shield 600 may comprise a tethering hole 601 for secure attachment and a tethering ribbon 602 that extends from this hole with an adjustable length, (e.g., ranging from 30 to 50 cm). Attached to the tethering ribbon 602 is a non-rigid retractor flap 603, with adjustable dimensions (e.g., a width ranging from 0.5-1.5 cm and a height ranging from 1.5-2.5 cm) to accommodate various surgical requirements. The non-rigid retractor flap 603 connects to a string 604, which leads to an anchoring barb 605. The anchoring barb 605 may provide a surface to abut against one side of a biological tissue (e.g., annulus 801 of a disc, as shown in FIG. 8C and FIG. 9). The string 604 and anchoring barb 605 may together form a T-shape configured to fit into a barb loading portal of a dura shield deployment device (e.g., a barb loading portal 704 of FIG. 7). A length of the string 604 may reflect a thickness of the biological tissue (e.g., a thickness of annulus) such that upon deployment, the anchoring barb 605 is secured in place relative to the biological tissue without extending beyond the thickness of the biological tissue.

FIG. 7 shows an example of a dura shield applicator 700, featuring a button 701 that, if engaged, actuates an actuation plunger 702. This action advances the dura shield 600 from a barb loading portal 704 toward the biological tissue. The hand back stop 703, situated between the button 701 and the barb loading portal 704, provides ergonomic support to prevent slippage during use. A housing 706 encases a barb tail clearance channel 705 and tapered end 707 near the needle 708, which aids in transitioning the housing 706 to a smaller diameter of the needle 708. The needle 708 is coaxially housed within a cannula 709 (e.g., a tube), allowing for retraction and extension beyond the cannula 709's distal end to facilitate tissue penetration. The cannula 709 serves to guide the dura shield 600 from the barb loading portal 704 to a deployment site, aligning with the needle 708 to deliver the dura shield 600 into the biological tissue.

FIG. 8A shows the dura shield applicator 700 prior to loading the dura shield 600. The anchoring barb 605 of the dura shield 600 is prepared to align with and enter the barb loading portal 704 of the dura shield applicator 700. In FIG. 8B, the anchoring barb 605 is inside the barb tail clearance channel 705, ready to be maneuvered through the barb tail clearance channel 705 as the string 604 extends from the side slit 710 of the housing 706. The subsequent figures, FIG. 8C and FIG. 8D, show a progression of the anchoring barb 605, along with the non-rigid retractor flap 603 and the tethering ribbon 602, moving towards the dura shield applicator 700's end, where the cannula 709 is located.

In FIG. 8D, upon pressing the button 701, the actuation plunger 702 propels the needle 708 beyond the cannula 709's distal end, causing puncturing of a hole into the biological tissue, such as the annulus 801. The anchoring barb 605 is then guided towards the needle 708's tip and anchored into the hole, securing the dura shield 600 in place relative to the biological tissue.

FIG. 9 shows an example of a dura shield applied in Lateral Lumbar Interbody Fusion (LLIF) or Oblique Lateral Lumbar Interbody Fusion (OLLIF) procedures. LLIF procedure involves accessing the spinal column 900 from the side (laterally) of a patient's body. OLLIF procedure also involves a lateral access, but an angle is more oblique rather than directly from the side. The spinal column 900 comprises intervertebral discs 901 featuring an annulus fibrosus (e.g., annulus 801), which constitutes the outer layer of the intervertebral discs 901. The spinal column 900 further comprises the vertebral bodies 902 and the nerve roots 903 that branch out from the spinal cord and pass through the vertebral bodies 902. During the LLIF or OLLIF procedures, the dura shield 600, or specifically its non-rigid retractor flap 603, may be positioned to safeguard areas of the nerve roots 903 and the intervertebral discs 901. An implant 904 (e.g., a spacer or fusion device) may be inserted between the vertebral bodies 902.

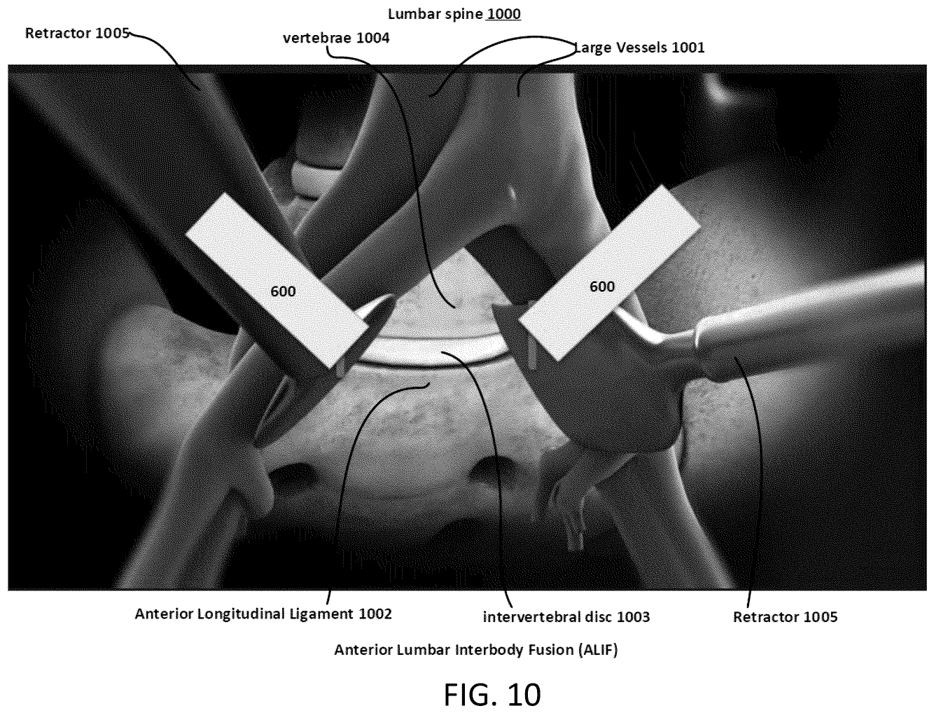

FIG. 10 shows an example of a dura shield applied in Anterior Lumbar Interbody Fusion (ALIF) procedure. During ALIF procedure, the lumbar spine 1000 may be accessed from the anterior, where the anterior longitudinal ligament 1002 is located, to remove a damaged intervertebral disc (e.g., intervertebral disc 1003). Then, two or more vertebrae 1004 may be fused using a spacer or implant. During ALIF procedure, the dura shield 600, particularly its non-rigid retractor flap 603, may be positioned to protect areas of blood vessels (e.g., large blood vessels 1001) situated anterior to the lumbar spine 1000. Retractors 1005 may be used to hold the large blood vessels 1001 aside and provide clear access to the surgical area.

According to the present disclosure, an apparatus may comprise a housing defining a barb loading portal configured to receive a dura shield, a hand back stop located on one end of the housing, the hand back stop configured to prevent slippage of a user's hand during operation of the apparatus, an actuation plunger movably disposed within the housing, wherein the actuation plunger is configured to advance the dura shield from the barb loading portal to a biological tissue, and a button coupled to the actuation plunger, wherein the button is configured to cause, upon engagement of the button, forward movement of the actuation plunger and delivery of the dura shield into the biological tissue.

The housing may further comprise a barb tail clearance channel aligned with the barb loading portal, and the barb tail clearance channel is configured to accommodate a tail portion of the dura shield upon an advancement of the dura shield by the actuation plunger.

The apparatus may further comprise a needle configured to penetrate the biological tissue, wherein another end, opposite to the one end, of the housing is coupled to the needle.

The hand back stop comprises a protrusion extending laterally from the housing, the protrusion having a curved profile conforming to a contour of a user's palm or fingers to facilitate a grip and prevent slippage during operation of the apparatus, and wherein the hand back stop is located between the barb loading portal and the button.

The housing comprises a tapered end proximate to a needle, the tapered end configured to provide a transition from a larger diameter of the housing to a smaller diameter of the needle.

The apparatus may further comprise a cannula attached to other end of the housing and aligned with the barb loading portal, wherein the cannula is configured to guide the dura shield from the barb loading portal to a distal end of the cannula.

The apparatus may further comprise a needle disposed within the cannula and configured to extend beyond the distal end of the cannula to penetrate the biological tissue, and a barb tail clearance channel located within the housing and aligned with the cannula, configured to accommodate a tail portion of the dura shield upon an advancement of the dura shield through the cannula by the actuation plunger.

The apparatus may further comprise a needle being coaxially disposed within the cannula and retractable relative to the cannula, wherein the cannula is configured to guide the dura shield from the barb loading portal to the distal end of the cannula for delivery into the biological tissue, wherein the needle is configured to create an initial penetration into the biological tissue, and cause the dura shield to be subsequently guided through the needle and into the biological tissue via the cannula.

The dura shield comprises an elongated body with an anchoring barb for anchoring within the biological tissue, the elongated body being configured to be slidably received within the cannula for deployment into the biological tissue.

The elongated body comprises a non-rigid retractor flap coupled to the anchoring barb, wherein the non-rigid retractor flap has a width of approximately 1 cm and a height of approximately 2 cm.

According to the present disclosure, an apparatus comprising a non-rigid retractor flap configured to protect a patient's dura during an interbody procedure, an anchoring barb coupled to the non-rigid retractor flap and configured to secure the non-rigid retractor flap to a biological tissue, and an applicator comprising a button and configured to receive, via a barb loading portal of the applicator, the anchoring barb along with the non-rigid retractor flap, and move forward and deliver the anchoring barb along with the non-rigid retractor flap into the biological tissue, wherein the button is configured to cause, upon engagement of the button, forward movement and delivery of the anchoring barb along with the non-rigid retractor flap into the biological tissue.

The applicator comprises a hand back stop configured to prevent slippage of a user's hand during operation of the applicator, and an actuation plunger configured to advance the anchoring barb from the barb loading portal, wherein the button is coupled to the actuation plunger, wherein pressing the button causes the actuation plunger to move forward and deliver the anchoring barb along with the non-rigid retractor flap into the biological tissue.

The applicator comprises a barb tail clearance channel aligned with the barb loading portal, the barb tail clearance channel configured to accommodate a tail portion of the anchoring barb as the anchoring barb is advanced toward the biological tissue.

The applicator comprises a needle configured to create an initial penetration into the biological tissue, and cause the anchoring barb to be subsequently guided through the needle and into the biological tissue.

The anchoring barb comprises a T-shape configuration, with a cross portion the T-shape providing a surface to abut against one side of the biological tissue and a tail portion of the T-shape extending through and corresponding in length to a thickness of the biological tissue, such that upon deployment, the anchoring barb is secured in place relative to the biological tissue without extending beyond the thickness of the biological tissue.

The apparatus may further comprise a tethering ribbon extending longitudinally from the non-rigid retractor flap, the tethering ribbon having a length between 30 to 50 cm to facilitate manipulation or positioning of the anchoring barb during deployment, wherein the tethering ribbon comprises a tethering hole.

According to the present disclosure, a method for delivering a dura shield into a biological tissue, the method comprising receiving the dura shield into a barb loading portal defined by a housing of an applicator, puncturing a hole into the biological tissue at a desired location on the biological tissue by pushing a needle disposed within a cannula coupled to the housing, and pressing a button, coupled to an actuation plunger movably disposed within the housing, to cause forward movement of the actuation plunger, advance the dura shield guided by the cannula, and drive the dura shield through the hole.

The method, wherein a barb tail clearance channel is located within the housing and aligned with the cannula, the method further comprising accommodating a tail portion of the dura shield as the dura shield is advanced through the cannula by the actuation plunger.

The method may further comprise positioning the needle at the desired location on the biological tissue, and after the pressing the button, withdrawing the needle from the biological tissue while leaving the dura shield in place.

The dura shield is delivered during an interbody procedure involved with at least one of: Transforaminal Lumbar Interbody Fusion, wherein the dura shield is deployed to protect dura mater or nerve roots while accessing disc space, Anterior Lumbar Interbody Fusion, wherein the dura shield is deployed to protect blood vessels, Lateral Lumbar Interbody Fusion wherein the dura shield is deployed to protect muscle or femoral nerve, anterior cervical discectomy and fusion, wherein the dura shield is deployed to protect esophagus, trachea, carotid artery, or venous network, corpectomy or vertebral column resection (VCR), wherein the dura shield is deployed to protect lung tissue when navigating through a thoracic cavity, pedicle subtraction osteotomy or VCR performed from a posterior, wherein the dura shield is deployed to protect dura mater or blood vessels, or spine surgery, neurosurgery, or general orthopedic surgery, wherein the dura shield is deployed to protect nerve tissue or blood vessels.

Although the present disclosure has been described in certain specific examples, many additional modifications and variations would be apparent to those skilled in the art. In particular, any of the various processes described above can be performed in alternative sequences and/or in parallel (on the same or on different computing devices) in order to achieve similar results in a manner that is more appropriate to the requirements of a specific application. It is therefore to be understood that the present disclosure can be practiced otherwise than specifically described without departing from the scope and spirit of the present disclosure. Thus, examples of the present disclosure should be considered in all respects as illustrative and not restrictive. It can be evident to the annotator skilled in the art to freely combine several or all of the examples discussed here as deemed suitable for a specific application. Throughout this disclosure, terms like “advantageous,” “exemplary,” or “preferred” indicate elements or dimensions which are particularly suitable (but not essential) to the disclosure or an example thereof, and can be modified wherever deemed suitable by the skilled annotator, except where expressly required. Accordingly, the scope of the disclosure should be determined not by the examples illustrated, but by the appended claims and their equivalents.

1-20. (canceled)

21. An apparatus comprising:
a housing defining a barb loading portal configured to receive an anchoring barb of an anatomical shield;
a handle located on one end of the housing, the handle configured to be gripped by a user's hand during operation of the apparatus;
a driver movably disposed within the housing, wherein actuation of the driver is configured to advance the anchoring barb of the anatomical shield from the barb loading portal to a biological tissue; and
an actuator coupled to the driver, wherein the actuator is configured to cause, upon actuation of the actuator, forward movement of the driver and delivery of the anchoring barb of the anatomical shield into the biological tissue.

22. The apparatus of claim 21, further comprising a tip configured to penetrate the biological tissue.

23. The apparatus of claim 21, wherein the handle comprises a protrusion extending laterally from the housing, the protrusion having a curved profile conforming to a contour of a user's palm or fingers to facilitate a grip and prevent slippage during operation of the apparatus, and wherein the handle is located between the barb loading portal and the actuator.

24. The apparatus of claim 21, wherein the housing comprises a tapered end proximate to a tip.

25. The apparatus of claim 21, further comprising a cannula aligned with the barb loading portal, wherein the cannula is configured to guide the anatomical shield from the barb loading portal to a distal end of the cannula.

26. The apparatus of claim 25, further comprising:
a needle disposed within the cannula and configured to extend beyond the distal end of the cannula to penetrate the biological tissue; and
a barb tail clearance channel located within the housing and aligned with the cannula, configured to accommodate a tail portion of the anatomical shield upon an advancement of the anatomical shield through the cannula by the driver.

27. A system comprising:
an anatomical shield comprising:
a retractor flap configured to protect a patient's dura during an interbody procedure; and
an anchoring barb coupled to the retractor flap and configured to secure the retractor flap to a biological tissue; and
an applicator comprising an actuator and configured to:
receive, via a portal of the applicator, the anchoring barb; and
deploy the anchoring barb into the biological tissue, wherein the actuator is configured to cause, upon actuation of the actuator, forward movement and delivery of the anchoring barb into the biological tissue.

28. The system of claim 27, wherein the applicator comprises:
a handle configured to be gripped by a user's hand during operation of the applicator; and
a driver configured to advance the anchoring barb from the portal, wherein the actuator is coupled to the driver, wherein actuating the actuator causes the driver to move forward and deliver the anchoring barb into the biological tissue.

29. The system of claim 27, wherein the applicator comprises:
a tip configured to create an initial penetration into the biological tissue, and cause the anchoring barb to be subsequently guided through the tip and into the biological tissue.

30. The system of claim 27, wherein the retractor flap is non-rigid.

31. The system of claim 27, wherein the anchoring barb comprises a T-shape configuration.

32. The system of claim 31, wherein a cross portion of the T-shape provides a surface to abut against one side of the biological tissue and a tail portion of the T-shape extending through and corresponding in length to a thickness of the biological tissue, such that upon deployment, the anchoring barb is secured in place relative to the biological tissue without extending beyond the thickness of the biological tissue.

33. The system of claim 27, further comprising a tethering ribbon extending longitudinally from the retractor flap.

34. The system of claim 33, wherein the tethering ribbon has a length between 30 to 50 cm to facilitate manipulation or positioning of the anchoring barb during deployment, wherein the tethering ribbon.

35. The system of claim 33, wherein the tethering ribbon comprises a tethering hole.

36. A method for delivering an anatomical shield into a biological tissue, the method comprising:
receiving an anchoring barb of the anatomical shield into a portal defined by a housing of an applicator;
puncturing a hole into the biological tissue at a desired location on the biological tissue by advancing a tip of the applicator into the biological tissue; and
actuating an actuator coupled to a driver movably disposed within the housing, to cause forward movement of the driver and drive the anatomical shield through the hole.

37. The method of claim 36, further comprising:
positioning the tip at the desired location on the biological tissue; and
after the actuating the actuator, withdrawing the tip from the biological tissue while leaving the anatomical shield in place.

38. The method of claim 36, wherein the anatomical shield is delivered during an interbody procedure comprising at least one of:
Transforaminal Lumbar Interbody Fusion, wherein the anatomical shield is deployed to protect dura mater or nerve roots while accessing disc space;
Anterior Lumbar Interbody Fusion, wherein the anatomical shield is deployed to protect blood vessels;
Lateral Lumbar Interbody Fusion wherein the anatomical shield is deployed to protect muscle or femoral nerve;
anterior cervical discectomy and fusion, wherein the anatomical shield is deployed to protect esophagus, trachea, carotid artery, or venous network;
corpectomy or vertebral column resection (VCR), wherein the anatomical shield is deployed to protect lung tissue when navigating through a thoracic cavity;
pedicle subtraction osteotomy or VCR performed from a posterior, wherein the anatomical shield is deployed to protect dura mater or blood vessels; or
spine surgery, neurosurgery, or general orthopedic surgery, wherein the anatomical shield is deployed to protect nerve tissue or blood vessels.

39. The method of claim 37, wherein the anchoring barb comprises a T-shape configuration, wherein a cross portion of the T-shape provides a surface to abut against one side of the biological tissue and a tail portion of the T-shape extending through and corresponding in length to a thickness of the biological tissue, such that upon deployment, the anchoring barb is secured in place relative to the biological tissue without extending beyond the thickness of the biological tissue.

40. The method of claim 37, wherein the anatomical shield comprises a retractor flap configured to protect a patient's dura during an interbody procedure and a tethering ribbon extending longitudinally from the retractor flap.
